(19) United States
(12) Patent Application Publication
(10) Pub. No.: US 2023/0046857 A1
(43) Pub. Date: Feb. 16, 2023

(54) SYSTEM AND METHOD FOR MEASURING A PHYSICAL PARAMETER IN A GASEOUS SAMPLE

(71) Applicant: Kamal Mahajan, Ronkonkoma, NY (US)

(72) Inventor: Kamal Mahajan, Ronkonkoma, NY (US)

(21) Appl. No.: 17/402,523

(22) Filed: Aug. 14, 2021

(51) Int. Cl. G01N 22/04; G01N 33/00

(52) U.S. Cl. CPC G01N 22/04; G01N 33/0027

(57) ABSTRACT

A system and method for measuring a parameter in a gaseous sample is disclosed herein. A signal generator generates a first signal. The first signal is sent through the gaseous sample, enclosed in an airtight chamber, via a transmitter and received via a receiver. The signal is then processed to measure the delay in transmission along a predetermined distance. The delay in the transmission of the signal from the transmitter to the receiver through the sample along a predetermined distance gas gives a measure of the parameter being measured within the gaseous sample. Examples of parameters that can be measured include humidity, temperature, air quality, pressure, and a quantity of a specific contaminant.

### FIELD OF INVENTION

The present disclosure relates to a system and a method for measuring a specific physical parameter in a gaseous sample.

### BACKGROUND

Measurement of a particular parameter in a fluid medium, such as air, typically requires the use of a dedicated sensing device. The sensing device typically includes a sensor that senses the desired parameter and provides a relevant signal to a controller and actuator which then converts the signal into respective readings of the measured parameter through a system of feedback and control. The readings are then communicated to the user through a display. An example of such is the sensing of atmospheric pressure using a barometer where the parameter, air pressure, is sensed as a measure of the weight of surrounding air on mercury within a column, which serves as the sensing material. The signal which is the height of mercury in the column is then converted and displayed as pressure units. Similarly, the thermal expansion of a fluid within a glass column is used as a measure of temperature in a thermometer. Examples of such devices for sensing parameters in fluids include those disclosed in U.S. Published Patent Application 2010/0307238 to Van Popta et al., U.S. Pat. No. 9,470,585 to Hong et al., and U.S. Pat. No. 8,326,568 to Hsu et al.

A disadvantageous aspect, however, of the typical sensing devices is the time required by the device to arrive at the conclusive measurement of the desired parameter. This is often due to the intrinsic limitation of the mechanism by which the conventional sensing device operates. For example, a hygrometer may include a metal oxide or dielectric material placed between electrodes. As the moisture in the air, hence humidity, changes, capacitance changes in response. The change in the capacitance is affected by the rate of moisture exchange between the sensing material and the environment. This is a limiting factor in the performance of such sensors. Although response times can be from several seconds down to fractions of a second, this can take too long for some applications. As an example, a hygrometer may be employed in an airplane to measure in real time relative humidity to determine if there is a microburst ahead while landing. The sooner information can be detected of a potential danger, the more likely disaster can be averted. Where such sensors or detectors are implemented within a control system fast response is required to ensure fast feedback and effective regulation of the set parameter.

A further disadvantage of the conventional measurement devices for gaseous sample parameters is the material properties limitation. For certain conventional sensing materials, the environment of measurement alters the intrinsic properties of the material. This alteration in the material properties affects the measurement. This demands for more complex implementation processes to ensure that the device provides accurate readings or otherwise in a sensing device of limited capability.

### SUMMARY

The present disclosure is of a system and method for measuring a physical parameter in a gaseous sample. The system comprises a processor connected to a voltage control oscillator (VCO) within a phase locked loop (PLL) and a memory storing a set of program modules executable by the processor. The voltage control oscillator herein refers to an electronic oscillator whereby the frequency is determined by the input voltage. The term phase locked loop within this disclosure refers to a control system generating an output signal whereby the phase of the signal is dependent on the input signal. In such a control system the phase detector compares the input and output signal phase. In the conventional system the signals from the oscillator are adjusted in order to match the signals. In the present disclosure the phase locked loop is executed in a different manner as disclosed herein. The system is configured to generate, via a voltage control oscillator that is coupled to the processor and phase locked loop, a first signal having a first frequency. In the current disclosure, the process for measuring a specific parameter begins when a specific voltage is applied to the voltage control oscillator. This causes the first signal to be transmitted through the gaseous sample for which the desired parameter is to be measured. The transmitted first signal is received via a receiver, and the receiver generates a received signal wherein the received signal is the transmitted first signal accompanied with the transmission delay while the signal is passing through the gaseous sample. In one embodiment, the receiver is spaced apart from the transmitter by a predetermined first distance. The received signal is supplied to a phase locked loop, wherein the phase locked loop is coupled to the processor, the receiver, and the voltage control oscillator. The phase locked loop is configured to receive the received signal and a reference signal from the voltage control oscillator. The received signal and the reference signal are then matched to generate a synchronized signal and supply the synchronized signal to the processor. A processor, which can be a digital signal processor or other types of processors, is configured to analyze the synchronized signal and the reference signal to compute the delay in the transmission of the first signal from the transmitter to the receiver through the gaseous sample, and determine, based on the transmission delay, a measure of the parameter in the gaseous sample.

The present disclosure also presents a method for measuring a parameter in a gaseous sample which implements the system disclosed herein.

### BRIEF DESCRIPTION OF THE DRAWINGS

FIG. 1 shows a schematic block diagram of a system for measuring a parameter in a sample, according to one embodiment of the present disclosure.

FIG. 2 shows a flow diagram of a method for measuring a parameter in a sample, according to one embodiment of the present disclosure.

### DETAILED DESCRIPTION

Example embodiments of the disclosure now will be described more fully hereinafter with reference to the accompanying drawings, in which example embodiments are shown. The concepts discussed herein may, however, be embodied in many different forms and should not be construed as limited to the example embodiments set forth herein; rather, these embodiments are provided so that this disclosure will be thorough and complete, and will fully convey the scope to those of ordinary skill in the art. Like numbers refer to like elements but not necessarily the same or identical elements throughout.

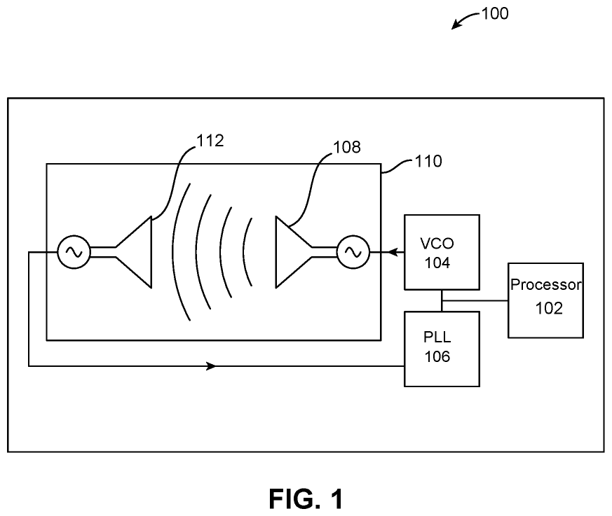

Referring to FIG. 1, a schematic block diagram of a system for measuring a parameter in a sample, according to a first embodiment of the present disclosure, is illustrated. The system 100 comprises a processor 102. In one embodiment, the processor 102 is of the type having an integrated memory. The integrated memory is configured to store a set of program modules executable by the processor 102 to operate the system 100, which are disposed (e.g., embedded) on a non-transitory storage medium. In an embodiment, the processor 102 includes more than one computing device, such as a digital signal processor (DSP) for signal processing and an additional microprocessor for program logic.

In one implementation, the processor 102 includes interface(s), one or more processor(s), and a memory coupled to the processor(s). In an embodiment, the interfaces may include a variety of software and hardware interfaces, for example, interfaces for peripheral device(s), such as an external memory, a display, and a printer. The memory may include any computer-readable storage medium known in the art including, for example, volatile memory such as static random-access memory (SRAM) and dynamic random-access memory (DRAM), and/or non-volatile memory, such as read only memory (ROM), erasable programmable ROM, flash memories, hard disks, optical disks, and magnetic tapes. Instructions for operation of the processor 102 are stored on a non-transitory computer-readable medium. As used herein, non-transitory computer-readable media comprises all computer-readable media except for a transitory, propagating signal.

The system 100 further comprises a voltage control oscillator 104 (alternatively referred to as VCO 104) and a phase locked loop 106 (PLL 106). The processor 102 is communicatively coupled to the voltage control oscillator 104 and the phase locked loop 106.

In accordance with an embodiment of the present disclosure, the VCO 104 is configured to generate a first signal having a first frequency. In one embodiment, the first frequency is about 5.9 GHz. It is to be noted that the frequency of the first signal generated via the VCO is generated based on the input control voltage supplied to the VCO. In one embodiment, the control voltage supplied to the VCO 104 is such that the first signal generated by the VCO 104 has a frequency of 5.9 GHz. However, the input voltage can vary such that the frequency can be of any value that is feasible for the execution of the process.

The VCO can also be substituted with any such device that transmits signals and communicates with the processor and phase locked loop in accordance with the current invention (one which will result in a delay in signal corresponding with varying parameters such as humidity, level of contamination, and pressure).

The system 100 further comprises a transmitter 108 coupled to the VCO 104. The transmitter 108, in accordance with one embodiment of the present disclosure, is a radiofrequency transmitter. In other embodiments of the invention the transmitter can be of other forms transmitting any form of signal that can be received by the receiver. According to one implementation, the transmitter 108 is installed in a airtight chamber 110 of fixed volume and dimensions. The airtight chamber 110, in accordance with one embodiment, is the space where the gaseous sample is introduced, wherein a desired parameter of the gaseous sample is to be measured. The airtight chamber 110 will be covered in an RF shielding material to ensure that all the signal from the transmitter reaches the receiver without environmental interference. The first signal generated by the VCO 104 is then transmitted through the sample air present within the airtight chamber 110 and received via a receiver 112. The receiver 112 is also located within the airtight chamber 110. More specifically, the transmitter 108 and the receiver 112 can be placed at the opposite ends of the airtight chamber 110 and separated by a first predetermined distance. In one embodiment, the first distance can range from about 0.5 inches to about 3 inches, although other distances may suffice.

The transmitted first signal is received by the receiver 112, and the receiver 112 generates a received signal, wherein the received signal is the transmitted first signal accompanied with the transmission delay while the signal is passing through the gaseous sample. The receiver 112 is coupled to the phase locked loop 106. The receiver sends the received signal to the phase locked loop 106. The phase locked loop 106 is also coupled to the VCO 104 such that the VCO 104 supplies a reference signal to the phase locked loop 106 (alternatively referred to as PLL 106). As the PLL 106 receives the received signal from the receiver and the reference signal from the transmitter, the received signal is adjusted by the PLL 106 to generate a synchronized signal, which may then be used as the signal having the required control voltage to continue the operation of the VCO 104. In one embodiment, the reference signal has the same frequency and phase as that of the first signal.

The synchronized signal is then supplied to the processor 102. The synchronized signal is analyzed by the processor 102 to extract the information pertaining to the transmission delay in the transmission of the first signal from the transmitter 108 to the receiver 112. In one embodiment, the transmission delay of the signal from the transmitter to the receiver is affected by the density of the gaseous sample, wherein the density of the gaseous sample is a factor that is translated into a measurement of the desired parameter within the gaseous sample due to the direct or derived relationship between the density and other parameters of the gaseous sample. Some examples of the parameters that can be measured using the system 100 include, but are not limited to, at least one of humidity, temperature, air quality, pressure, and quantity of a specific contaminant.

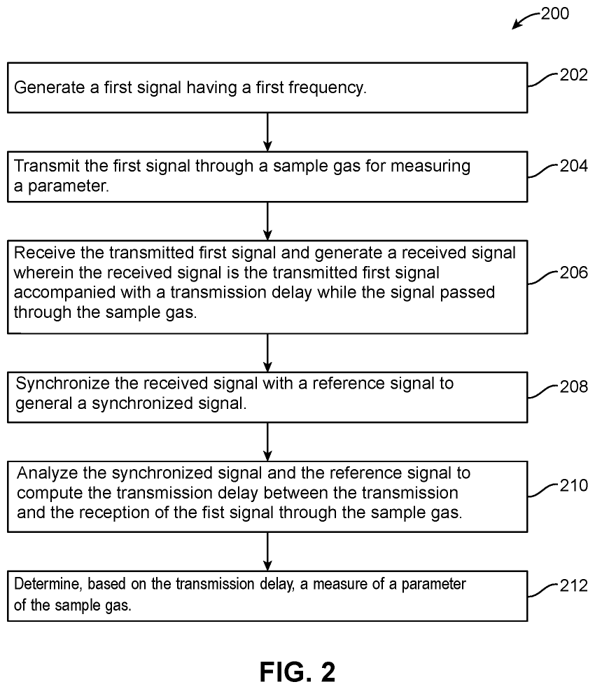

Referring to FIG. 2, a flow diagram of a method 200 for measuring a parameter in a sample, according to a first embodiment of the present disclosure, is illustrated. The order in which the method 200 is described is not intended to be construed as a limitation, and any number of the described method blocks can be combined in any order to implement the method or any alternative methods so long as any such system involves the transmission of a signal within an airtight chamber to measure a parameter of gaseous sample. Additionally, individual blocks may be deleted from the method without departing from the spirit and scope of the subject matter described herein. Furthermore, the method can be implemented in any suitable hardware, software, firmware, or combination thereof.

At step 202, the method 200 includes generating a first signal having a first frequency. In accordance with an embodiment of the present disclosure, this step is performed by the VCO 104.

At step 204, the method 200 includes transmitting the first signal through the gaseous sample for measuring the parameter. In accordance with an embodiment of the present disclosure, the first signal is transmitted through the gaseous sample via the transmitter 108, wherein the transmitter 108 is coupled to the VCO 104. The gaseous sample can be a sample of a single gas or any mixture of gases with varying composition and chemistry. The gaseous sample may also have suspended particles such as dusts and other contaminants.

At step 206, the method 200 includes receiving the transmitted first signal and generating a received signal wherein the received signal is the transmitted first signal accompanied with the transmission delay while the signal is passing through the gaseous sample. In accordance with an embodiment of the present disclosure, the receiver 112 is configured to receive the transmitted first signal and thereafter generate the received signal.

At step 208, the method 200 includes synchronizing the received signal with a reference signal to generate a synchronized signal. In accordance with an embodiment of the present disclosure, the PLL 106 is configured to synchronize the received signal with the reference signal to generate a synchronized signal.

At step 210, the method 200 analyzes the synchronized signal and the reference signal to compute the transmission delay between the transmission and the reception of the first signal through the gaseous sample. At block 212, the method 200 includes determining, based on the transmission delay, a measure of the parameter in the gaseous sample. In accordance with one embodiment, the processor 102 is configured to analyze the synchronized signal and the reference signal to compute the transmission delay. Thereafter, the processor 102 determines the measurement of the desired parameter based on the transmission delay. The measurement of the desired parameter based on the transmission delay is then output in human perceptible form, such as by displaying the information on a computer screen or printing it using a printer.

Those skilled in the art will readily recognize, in light of and in accordance with the subject matter disclosed in the present disclosure, that any of the aforementioned method steps may be implemented using at least one of a wide variety of suitable processes and system modules or system components, and is not limited to any particular computer hardware, firmware, microcode, software, middleware, and the like. The programs useable to instruct the processor 102 to perform the method steps may be written in C, C#, C++, Java, Brew, or any other suitable programming language.

For any method steps described in the present disclosure that can be carried out on a processing machine or a computing machine, the processor 102 can, when appropriately configured or designed, serve as a computer in which those aspects of the invention may be embodied. Such processors referenced and/or described in this disclosure may be any kind of computer, either general purpose, or some specific purpose computer such as, but not limited to, a workstation, a mainframe, GPU, ASIC, etc. For example, the processor 102 can be a microprocessor having program code embedded thereon, or comprise a separate computer system, either standalone or networked. Where the processor 102 is networked, t processing can be performed on one or more processors, such as in accordance with a client/server approach.

### Example 1

An exemplary operation of the system 100 is described herein. The system 100, in accordance with the present implementation, is configured for measurement of humidity within a gaseous sample which can be air or any other mixture of gases or a single type of gas. The gaseous sample, the humidity of which is to be measured, is supplied in the airtight chamber 110. A first signal at about 5.9 GHz frequency is transmitted via the transmitter 108 through the gaseous sample that is introduced in the airtight chamber 110. The transmitted first signal is received by the receiver 112 as the signal passes through the sample air. As the transmitted first signal is received by the receiver 112, the receiver 112 generates a corresponding received signal. The received signal, in accordance with the present disclosure, is the first signal accompanied by the transmission delay in transmission of the first signal from the transmitter to the receiver.

The received signal is then supplied to the PLL 106. The PLL 106 also receives a reference signal from the VCO 104. The PLL 106 is configured to match the received signal and the reference signal to generate a synchronized signal. The synchronized signal is then analyzed by the processor 102 for extracting the information pertaining to the transmission delay. As mentioned previously, the transmission delay in this example, gives the measurement of the humidity in the gaseous sample.

For translating the transmission delay into the measurement of humidity, the system 100 needs to be initially calibrated for the same. More specifically, in accordance with one implementation of the present disclosure, a completely dry gaseous sample with 0% humidity may be introduced into the airtight chamber 110, and the transmission delay for the first signal to pass through the gaseous sample may be measured, using the aforementioned implementation. It is to be noted that for the purpose of calibration, a conventional humidity sensor may be used to confirm the humidity measure of the gaseous sample being introduced into the airtight chamber 110. An example of such a conventional humidity sensor is that disclosed in U.S. Pat. No. 5,844,138 to Cota, U.S. Published Patent Application 2010/0307238 to Van Popta et al., or any other types of commercially available humidity sensors that effectively measure humidity.

The transmission delay for the transmission of the first signal from the transmitter to the receiver through the completely dry sample air may be recorded. Similarly, the aforementioned calibration protocol may be repeated by incrementally increasing the humidity in the sample air being introduced in the airtight chamber 110, and thereafter recording the corresponding transmission delays. The increment may be by 1% or 10% relative humidity or any other increment such that the transmission delays are obtained for a range of humidity between 0% and 100%. These values are obtained while every other parameter such as sample composition, density, temperature and pressure are kept constant while only the humidity is varied. Once a table including a humidity measurement in percentage and the corresponding transmission delays (in picoseconds) is generated, the same may be stored in the integrated memory of the processor 102.

After the table of the humidity measurement in percentage and transmission delay is generated, the processor 102 can then be configured to extract the transmission delay information associated with the sample air from the synchronized signal and map the transmission delay value with the closest humidity measurement present in the table. As such, the system 100 is, in this manner, configured to measure the humidity of the sample air without the usage of any conventional analog humidity sensor after the generation of the relevant database. An advantageous aspect of eliminating the usage of the conventional analog humidity sensor is that the processing time is significantly reduced to being almost instantaneous as the speed of the signal transmission through the gaseous sample is within thousandths of a second. This permits usage in highly time sensitive and real time applications. It also significantly reduces the typical wait times in such applications as humidity probe calibration. The processor 102 only needs to perform one operation of mapping the transmission delay against humidity measurement within the table. As such, the system 100 may be made capable of providing virtually instantaneous measurement of a particular parameter in a gaseous sample. The processor speed therefore becomes the limiting factor in the response time rather than that of a sensing material which is significantly slowed by the mechanical interaction between the gaseous sample and the sensing material.

Another advantageous aspect of such a system for measuring a parameter in a gaseous sample, in accordance with the present disclosure, is that the system 100 may be configured to measure any relevant parameter in a fluid sample. More specifically, in the aforementioned example of the system 100, the system 100 is calibrated to measure humidity in a gaseous sample. However, the system 100 may also be configured to measure any other parameter, including but not limited to, temperature, pressure, air quality, quantity, any specific contaminant in an air sample, and so on.

### Example 2

A second exemplary operation of the system 100 is described herein. The system 100, in accordance with the present implementation, is configured for measurement of a specific contaminant within a sample of air. The sample air, the level of specific contamination of which is to be measured, is supplied in the airtight chamber 110. A first signal at about 5.9 GHz frequency is transmitted via the transmitter 108 through the sample air that is introduced in the airtight chamber 110. The transmitted first signal is received by the receiver 112 as the signal passes through the sample air. As the transmitted first signal is received by the receiver 112, the receiver 112 generates a corresponding received signal. The received signal, in accordance with the present disclosure, is the first signal accompanied by the transmission delay in transmission of the first signal from the transmitter to the receiver.

The received signal is then supplied to the PLL 106. The PLL 106 also receives a reference signal from the VCO 104. The PLL 106 is configured to match the received signal and the reference signal to generate a synchronized signal. The synchronized signal is then analyzed by the processor 102 for extracting the information pertaining to the transmission delay. As mentioned previously, the transmission delay gives the measurement of the contaminant in the gaseous sample.

For translating the transmission delay into the measurement of contamination, the system 100 needs to be initially calibrated for the same. More specifically, in accordance with one implementation of the present disclosure, a completely pure form of the gaseous sample with 0% contamination may be introduced into the airtight chamber 110, and the transmission delay for the first signal to pass through the gaseous sample may be measured, using the aforementioned implementation. The transmission delay for the transmission of the first signal from the transmitter to the receiver through the completely pure gaseous sample may be recorded. Similarly, the aforementioned calibration protocol may be repeated by incrementally increasing the concentration of the specific contaminant in the gaseous sample being introduced in the airtight chamber 110, and thereafter recording the corresponding transmission delays. The increment may be by 0.1%, 1% or 10% v/v or any other increment such that the transmission delays are obtained for a range of contaminations between 0% and 100%. These values are obtained with every other parameter such as humidity, gaseous sample composition, density, temperature, and pressure, kept constant while the level of contamination is varied. Once a table including contamination measurement in percentage and the corresponding transmission delays (in picoseconds) is generated, the same may be stored in the integrated memory of the processor 102.

After the table of the contamination measurement in percentage v/v and transmission delay is generated, the processor 102 can then be configured to extract the transmission delay information associated with the gaseous sample from the synchronized signal and map the transmission delay value with the closest % contamination measurement present in the database. As such, the system 100 is, in this manner, configured to measure the contamination in the gaseous sample without the usage of any conventional analog sensor after the generation of the relevant database.

### Example 3

A third exemplary operation of the system 100 is described herein, whereby the system 100, in accordance with the present implementation, is implemented within a mobile device and configured for measurement of a specific contaminant in the environment around the device within which it is implemented. To achieve this the system 100 is coupled with an automatic sampling system to periodically obtain sample air from the environment. An example of such an automatic air sampling system is presented in WO2003004996A3. This withdraws air from the surrounding environment and supplies the air sample to the airtight chamber 110. A first signal at about 5.9 GHz frequency is transmitted via the transmitter 108 through the sample air that is introduced in the airtight chamber 110. The transmitted first signal is received by the receiver 112 as the signal passes through the sample air. As the transmitted first signal is received by the receiver 112, the receiver 112 generates a corresponding received signal. The received signal, in accordance with the present disclosure, is the first signal accompanied by the transmission delay in transmission of the first signal from the transmitter to the receiver.

The received signal is then supplied to the PLL 106. The PLL 106 also receives a reference signal from the VCO 104. The PLL 106 is configured to synchronize the received signal and the reference signal to generate a synchronized signal. The synchronized signal is then analyzed by the processor 102 for extracting the information pertaining to the transmission delay. As mentioned previously, the transmission delay gives the measurement of the contaminant in the gaseous sample.

For translating the transmission delay into the measurement of contamination, the system 100 needs to be initially calibrated for the same. More specifically, in accordance with one implementation of the present disclosure, a completely pure gaseous sample with 0% contamination may be introduced into the airtight chamber 110, and the transmission delay for the first signal to pass through the gaseous sample may be measured, using the aforementioned implementation. The transmission delay for the transmission of the first signal from the transmitter to the receiver through the pure sample air may be recorded. Similarly, the aforementioned calibration protocol may be repeated by incrementally increasing the concentration of the specific contaminant in the gaseous sample being introduced in the airtight chamber 110, and thereafter recording the corresponding transmission delays. The increment may be by 0.1%, 1% or 10% v/v or any other increment. Such that the transmission delays are obtained for a range of contaminations between 0% and 100%. At constant transmission distance, the trajectory of the signal is affected by the properties of the medium through which it travels, in this case the gaseous sample. These values are obtained with every other parameter such as humidity, gaseous sample composition, density, temperature, and pressure, kept constant while the level of contamination is varied. Once a table including contamination measurement in percentage and the corresponding transmission delays (in picoseconds) is generated, the same may be stored in the integrated memory of the processor 102.

After the table of the contamination measurement in percentage v/v and transmission delay is generated, the processor 102 can then be configured to extract the transmission delay information associated with the sample air from the synchronized signal and map the transmission delay value with the closest % contamination measurement present in the database. As such, the system 100 is, in this manner, configured to measure the contamination in the gaseous sample without the usage of any conventional analog sensor after the generation of the relevant database.

Many modifications and other implementations of the disclosure set forth herein will be apparent having the benefit of the teachings presented in the foregoing descriptions and the associated drawings. Therefore, it is to be understood that the disclosure is not to be limited to the specific implementations disclosed and that modifications and other implementations are intended to be included within the scope of the appended claims. Although specific terms are employed herein, they are used in a generic and descriptive sense only and not for purposes of limitation.

What is claimed is:

1. A system for measuring a parameter in a gaseous sample, the system comprising:
a voltage control oscillator within a phase locked loop, a processor, and a non-transitory storage medium storing a set of program instructions executable by the processor to:
generate, via the voltage control oscillator that is coupled to the processor, a first signal having a first frequency;
transmit, via a transmitter that is coupled to the voltage control oscillator, the first signal through the gaseous sample for measuring the parameter;
receive, via a receiver, the transmitted first signal and generate a received signal wherein the received signal is the transmitted first signal accompanied with a transmission delay while the signal is passing through the gaseous sample, wherein the receiver is spaced apart from the transmitter by a first distance;
supply, the received signal to a phase locked loop, wherein the phase locked loop is coupled to the processor, the receiver, and the voltage control oscillator, the phase locked loop is configured to:
receive the received signal and a reference signal from voltage control oscillator;
match the received signal and the reference signal to generate a synchronized signal; and
supply the synchronized signal to the processor;
wherein the processor is configured to:
analyze the synchronized signal and the reference signal to compute the transmission delay in the transmission of the first signal from the transmitter to the receiver through the gaseous sample; and
determine, based on the transmission delay, a measure of the parameter in the gaseous sample.

2. The system according to claim 1, wherein the reference signal and the first signal are of the same phase and frequency.

3. The system according to claim 1, wherein the parameter is at least one of humidity, temperature, air quality, pressure, and quantity of a contaminant.

4. The system according to claim 1, wherein the parameter is a measurable characteristic of the gaseous sample.

5. The system according to claim 1, further comprising a substantially airtight chamber, wherein the gaseous sample is fed into the airtight chamber, and the transmitter and the receiver are positioned inside the airtight chamber.

6. The system according to claim 5, wherein the signal transmitted within the airtight chamber is any form of a signal such that a change in the parameter to be measured causes a detectable corresponding change in transmission delay between the receiver and the transmitter.

7. The system according to claim 1, wherein the storage medium includes data corresponding to a measure of a parameter and associated transmission delays.

8. The system according to claim 1, wherein the transmitter is a radio frequency transmitter, and the receiver is a radio frequency receiver.

9. The system according to claim 1, wherein the first distance ranges from about 0.5 inches to about 3 inches.

10. The system according to claim 1, wherein the gaseous sample is a gaseous phase of a single compound or element or a mixture of gases.

11. The system according to claim 1, wherein the voltage control oscillator is replaceable by any system which generates a signal when induced and is connectable with the phase locked loop and the processor.

12. The system according to claim 1, further including a display device that is configured to output the measure of the parameter in the gaseous sample in human perceptible form.

13. The system according to claim 12, wherein the display device is a screen.

14. A method for measuring a parameter in a gaseous sample, the method comprising:
generating a first signal having a first frequency;
transmitting the first signal through the gaseous sample for measuring the parameter;
receiving the transmitted first signal and generating a received signal wherein the received signal is the transmitted first signal accompanied with the transmission delay while the signal is passing through the gaseous sample;
synchronizing the received signal with a reference signal to generate a synchronized signal;
analyzing the synchronized signal and the reference signal to compute the transmission delay between the transmission and the reception of the first signal through the gaseous sample; and
determining, based on the transmission delay, a measure of the parameter in the gaseous sample.

15. The method according to claim 14, wherein the reference signal and the first signal are of the same phase and frequency.

16. The method according to claim 14, wherein the parameter is at least one of humidity, temperature, air quality, pressure, and a quantity of a specific contaminant.

17. The method according to claim 14, wherein the parameter relates to a measurable characteristic of the gaseous sample.

18. The method according to claim 14, wherein the gaseous sample comprises a gaseous phase of a single compound or element, or a mixture of gases.

19. The method according to claim 14, wherein the voltage control oscillator is replaceable by any device that generates a signal when induced and is connectable with the phase locked loop and the processor.

20. The method according to claim 14, further comprising:
outputting the measure of the parameter in the gaseous sample in human perceptible form.
